(19) United States
(12) Patent Application Publication
(10) Pub. No.: US 2006/0041292 A1
(43) Pub. Date: Feb. 23, 2006

(54) Single use ice pack and method

(75) Inventor: Michael L. Bowen, Mansfield, TX (US)

Correspondence Address: BAKER BOTTS L.L.P.;PATENT DEPARTMENT, 98 SAN JACINTO BLVD., SUITE 1500, AUSTIN, TX, 78701-4039, US

(73) Assignee: Advanced Materials, Inc.

(21) Appl. No.: 11/196,216

(22) Filed: Aug. 3, 2005

Related U.S. Application Data
(60) Provisional application No. 60/602,591, filed on Aug. 18, 2004.

(51) Int. Cl. A61F 7/00

(52) U.S. Cl. 607/114

(57) ABSTRACT

An ice pack including a bag having a containment section with an opening. At least one layer of adhesive material and preferably two respective layers of adhesive material may be disposed on adjacent portions of the opening. At least one removable liner and preferably two removable liners may be disposed on respective layers of the adhesive material. The ice pack may be satisfactory for a single use.

### RELATED APPLICATION

This application claims the benefit of U.S. Provisional Application No. 60/602,591, filed Aug. 18, 2004, the contents of which are hereby incorporated by reference in their entirety.

### TECHNICAL FIELD

The present invention is related to ice packs, and more particularly, to ice packs having a bag and closure mechanism satisfactory for a single use.

### BACKGROUND OF THE INVENTION

Ice packs are often used for cooling various objects, such as portions of a patient's body for medical reasons. Ice packs generally include a bag having a containment section with an associated opening or mouth. Ice may be inserted into the containment section of the bag through the mouth. Various types of closure mechanisms and devices have previously been used to seal the mouth of the bag after ice has been placed in the containment section. After sealing the mouth, the associated ice pack may be applied to a desired location on a patient.

Ice packs are preferably sealed so that ice and any liquid in the containment section will not run out of the ice pack. Some closure devices associated with prior ice packs close or seal the associated containment section using components such as stoppers and clips. Such components often add additional cost to manufacture an ice pack and may limit the quantity of ice which can be placed within an associated containment section. Various types of ties have also been used to seal or close ice packs. Ties may also be used to attach an ice pack to a patient's limb or other parts of a patient. Such ties are frequently not used due to time and effort required to effectively seal an ice pack having such ties. Many ice packs which are designed for multiple uses are often discarded after the first use, particularly when the first use is in a hospital or other medical facility.

### SUMMARY OF THE INVENTION

In accordance with teachings of the present invention, an ice pack may be formed with a bag having a first sidewall and a second sidewall sealed with each other to form a containment section and a mouth or opening communicating with the containment section. The mouth preferably includes a closure mechanism compatible with a single use of the ice pack. Each sidewall may be formed with one or more layers of material.

For some applications, the sidewalls may include a first layer or waterproof layer with a second layer or exterior layer formed from relatively soft, water absorbent material. At least one layer of adhesive material may be disposed on a portion of the mouth. For some applications, respective layers of adhesive material are preferably disposed on an interior portion of each sidewall adjacent to the mouth. At least one release liner may be disposed on adhesive material proximate the mouth. For some applications, a first release liner and a second release liner may be respectively disposed on a first layer and a second layer of adhesive material proximate the mouth. The first and second release liners may cooperate with each other to form a funnel type device to assist with placing ice within the containment section of an associated ice pack.

Technical benefits of the present invention include providing a relatively low cost ice pack with a reliable, easy to use closure mechanism. For some applications a disposable, low cost disposable funnel type device may be provided to assist with placing ice in the ice pack.

Further technical benefits of the present invention include providing a relative low cost closure mechanism which may be used to form a fluid barrier that substantially reduces or eliminates problems associated with leakage of water after an ice pack has been filled with ice. Such water leak is often a problem with conventional ice packs.

The present invention eliminates the need for relatively expensive clips or stoppers to close an ice pack. Eliminating hard plastic clips improves patient comfort during use of an ice pack having a soft, adhesive closure. The present invention eliminates ties and other types of closure mechanisms which are difficult and/or time consuming to use, particularly in a hospital emergency room. A single use ice pack may be particularly beneficial for use with patients in an isolation ward or similar facility. An associated single use or one time closure mechanism helps to prevent spread of contamination (germs and bacteria) by eliminating refills at an ice machine.

Ice packs incorporating teachings of the present invention may improve compliance with existing hospital rules and regulations concerning infection control and disposal of potentially contaminated materials while at the same time reducing costs. Ice packs may be formed in accordance with teachings of the present invention at one half to one third of the cost of conventional ice packs having the same general size and configuration.

One aspect of the present invention includes forming an ice pack by bonding or sealing portions of a first sidewall with a second sidewall to form a containment section having a mouth or opening. Ice may be placed in the containment section through the associated mouth. At least one adhesive layer may be disposed on one of the sidewalls proximate the associated mouth. The adhesive layer may contact adjacent portions of the other sidewall to form a fluid seal at the mouth after ice has been added to the containment section. For other applications respective adhesive layers may be disposed on each sidewall proximate the associated mouth. The adhesive layers may contact each other to form a fluid seal at the mouth after ice has been added to the containment section.

### BRIEF DESCRIPTION OF THE DRAWINGS

A more complete and thorough understanding of the present invention and advantages thereof may be acquired by referring to the following descriptions taken in conjunction with the accompanying drawings, in which like reference numbers indicate like features, and wherein:

FIG. 1 is a schematic drawing showing an isometric view of one embodiment of an ice pack incorporating teachings of the present invention;

FIG. 2 is a schematic drawing showing an exploded isometric view with portions broken away of various layers of material associated with the ice pack of FIG. 1;

FIG. 3 is a schematic drawing in section taken along lines 3-3 of FIG. 1;

FIG. 4 is a schematic drawing showing an isometric view with portions broken away of the ice pack of FIG. 1 being filled with ice in accordance with teachings of the present invention; and

FIG. 5 is a schematic drawing showing an isometric view with portions broken away of the ice pack of FIG. 1 during closure after being filled with ice in accordance with teachings of the present invention.

### DETAILED DESCRIPTION OF THE INVENTION

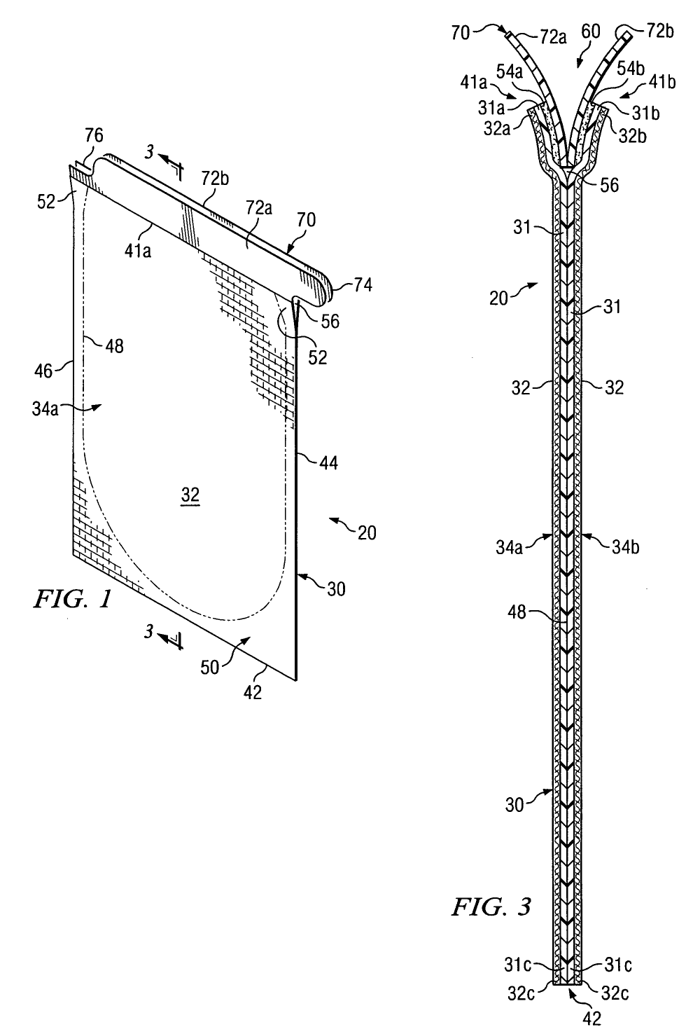

Various embodiments of the present invention and its advantages are best understood by reference to FIGS. 1-5 wherein like numbers refer to same and like parts.

FIGS. 1-5 show one example of an ice pack formed in accordance with teachings of the present invention. Ice pack 20 may be satisfactory for a single or one term use to apply cold therapy to a portion of a patient's body. Ice packs incorporating teachings of the present invention may be particularly useful when infection control is an important consideration.

For embodiments such as shown in FIGS. 1-5, ice pack 20 may be described as having a generally rectangular configuration. However, ice packs formed in accordance with teachings of the present invention may have a wide variety of other configurations (not expressly shown) such as circular, oval, square or any other appropriate configuration for applying to a patient.

Ice pack 20 may include bag 30 defined in part by containment section or cavity 40 and mouth or opening 60 communicating therewith. For some applications disposable funnel type device 70 may be included as part of ice pack 20. Ice pack 20 may include a one time closure mechanism incorporating teachings of the present invention. A layer of adhesive material and an attached release liner may be one example of such one time closure mechanism.

Bag 30 may be formed in part by a pair of sidewalls 34. For purposes of describing various features of the present invention sidewalls 34 have been designated as 34a and 34b. For some applications sidewalls 34a and 34b may be formed from the same materials with the same overall configuration and dimensions. For other applications sidewalls 34a and 34b may be formed with either different types of materials and/or with different configurations and dimensions. Ice packs may be formed in accordance with the teachings of the present invention with each sidewall having only one layer of material (not expressly shown) or with each sidewall having three or more layers of material (not expressly shown).

For some applications each sidewall 34 may include first layer or inner layer 31 and second layer or outer layer 32. For some applications first layer 31 may be formed from a wide variety of vinyl polymers such as relatively thin, low cost polyethylene film or any other waterproof material. Metallocene based polymers may also be used. Layer 31 may also be formed from various types of generally smooth, fluid impervious plastic films.

Second layer 32 may be formed from any soft, flexible, absorbent material satisfactory for contact with a patient's skin. For example layer 32 may be formed from various materials such as hydroentangled fibers and wood pulp. Polyester based fibers may also be used. Various types of commercially available woven and non-woven materials may be satisfactorily used to form second layer 32. Relatively low cost, inexpensive materials satisfactory for forming layers 31 and 32 are commercially available from a wide variety of manufacturers such as Polymer Group, Inc. (PGI).

For some applications layer 31 and associated layer 32 may be laminated with each other prior to forming bag 30. For example, a heat melt coating and/or film may be applied to one side of a layer of soft, absorbent woven or nonwoven material to produce a laminated sheet satisfactory for forming sidewalls 34. One side of the laminated sheet may be fluid impervious. The other side of the laminated sheet may be soft and absorbent. Sheets of laminated material are commercially available from several sources.

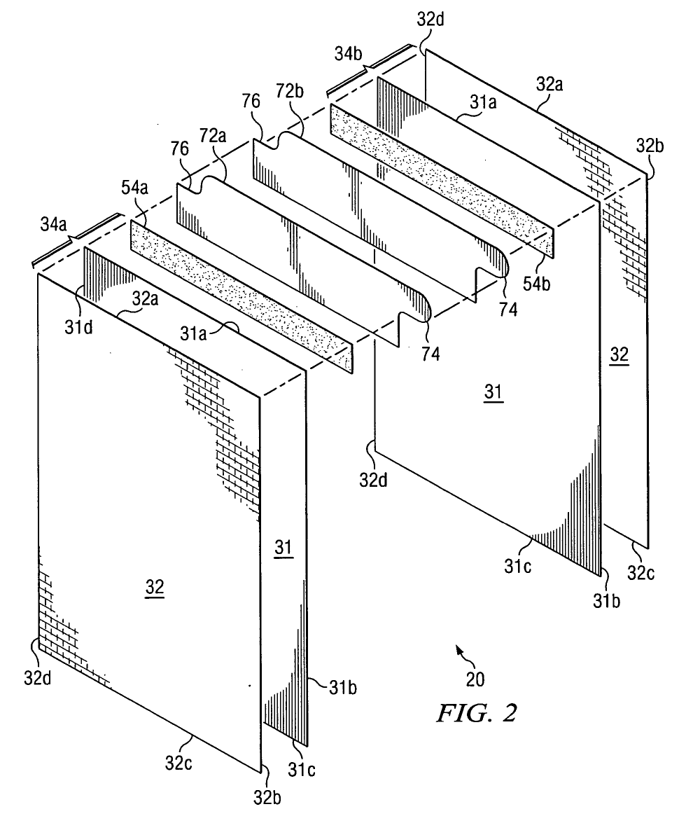

As shown in FIGS. 2 and 3, layers 31 and 32 may have generally rectangular configurations defined in part by respective edges 31a-31d and 32a-32d. See FIG. 2. Edges 32a and 32c for each layer 32 may extend generally parallel with each other between edges 32b and 32d. In a similar manner edges 31a and 31c for each layer 31 may extend generally parallel with each other between associated edges 31b and 31d. As previously noted for some applications, layers 31 and 32 may be laminated with each other prior to forming respective sidewalls 32a and 32b.

Bag 30 may be defined in part by first end 41 and second end 42 with lateral edges 44 and 46 extending therebetween. See FIGS. 1 and 3. Bag 30 includes mouth or opening 60 located at first end 41 for use in adding ice to containment section 40. Containment section 40 may be generally defined as a void space or cavity formed between sidewalls 34a, 34b, opening 60, respective sealed lateral edges 44, 46, and closed end 42.

First end 41 of bag 30 may include two portions designated 41a and 41b which cooperate with each other to provide mouth or opening 60. Lateral edge 44 of bag 30 may be formed by bonding or sealing edges 32b and 31b of sidewalls 34a and 34b with each other. In a similar manner, second end 42 or closed end 42 of bag 30 may be formed by bonding or sealing edges 32c and 31c of sidewalls 34a and 34b with each other. Lateral edge 46 may be formed by bonding or sealing edges 32d and 31d of sidewalls 34a and 34b with each other.

Various types of bonding and sealing techniques may be satisfactorily used to form fluid barrier 50 along the periphery of bag 30 adjacent to lateral edges 44 and 46 and end 42. For example, lateral edges 44, 46 and end 42 may be sealed or bonded using a heat sealing die (not expressly shown) to form fluid barrier 50 which extends along three sides of the periphery of bag 30. The dimensions and configuration of fluid barrier 50 may also be defined in part by dotted line 48 and adjacent lateral edges 44, 46 and closed end 42. Dotted line 48 also defines portions of containment section 40. Satisfactory fluid barriers may also be formed along the periphery of bag 30 using sonic welding techniques and/or RF energy bonding.

Respective adhesive layers 54a and 54b may be disposed on interior portions of each first layer 31 adjacent to respective first ends 41a and 41b proximate opening 60. Adhesive layers 54 may be formed from viscous, waterproof adhesive materials capable of filling any gaps associated with sealing edges 41a and 41b with each other and sealing with adjacent portions of lateral edges 44 and 46. Adhesive layers 54a and 54b may be used to permanently close or seal opening 60 after filling containment section 40 with ice. Various types of acrylic based adhesive materials and rubber based adhesive materials may be satisfactorily used to form adhesive layers 54a and 54b.

Release liners 72a and 72b are preferably attached with respective adhesive layers 54a and 54b. See FIGS. 2 and 3. For some applications, a single release liner may be satisfactorily used. However, release liners 72a and 72b cooperate with each other to provide a disposable funnel type device to facilitate adding ice to containment section 40 of bag 30. See FIG. 4. Release liners 72a and 72b may be formed from paper or other suitable plastic films. In addition to forming disposable funnel type device 70, release liners 72a and 72b cooperate with each other to protect associated adhesive layers 54a and 54b during fabrication of ice pack 20, storage prior to use and while adding ice to containment section 40.

For some applications, a single adhesive layer 54 may be placed on an interior portion of either sidewall 34a or 34b adjacent to mouth 60. A single release liner 72 may be placed on the associated adhesive layer 54. However, using two adhesive layers 54a and 54b in combination with release liners 72a and 72b will substantially improve the resulting fluid tight seal formed after ice has been placed within containment section 40.

Bag 30 may be described as having a pair of “flaps” located adjacent to opening 60. The first flap includes adhesive layer 54a, edge 41a, adjacent portions of associated layer 31 and adjacent portions of associated layer 32. The second flap includes adhesive layer 54b, edge 41b, adjacent portions of associated layer 31 and adjacent portions of associated layer 32. Release liner 72a may be attached to the first flap. Release liner 72b may be attached to the second flap.

For some applications portions of fluid barrier 50 adjacent to respective edges 41a and 41b may have an increased width. For embodiments such as shown in FIG. 1, the increased width portions of fluid barrier 50 have been designated as 52. Increased width portions 52 of fluid barrier 50 cooperate with adhesive layers 54a and 54b to form a fluid tight seal in any gaps which may be formed adjacent to edges 41a, 41b and other portions of the closure mechanism for mouth 60.

During the fabrication of ice pack 20 release liner 72a and 72b may result in the formation of the gap between adjacent portions of sidewalls 34a and 34b proximate mouth 60. The resulting gap may correspond approximately with the combined thickness of release liner 72a and 72b. By using relatively viscous adhesive materials to form adhesive layers 54a and 54b any associated gaps will be generally filled when adhesive layers 54a and 54b contact each other. Rubber based pressure sensitive adhesive is one example of such viscous adhesive materials. As discussed later in more detail, combining adhesive layers with heat sealing the periphery of a bag in accordance with teachings of the present invention substantially eliminates or reduces any gaps associated with closure of the bag after having been filled with ice.

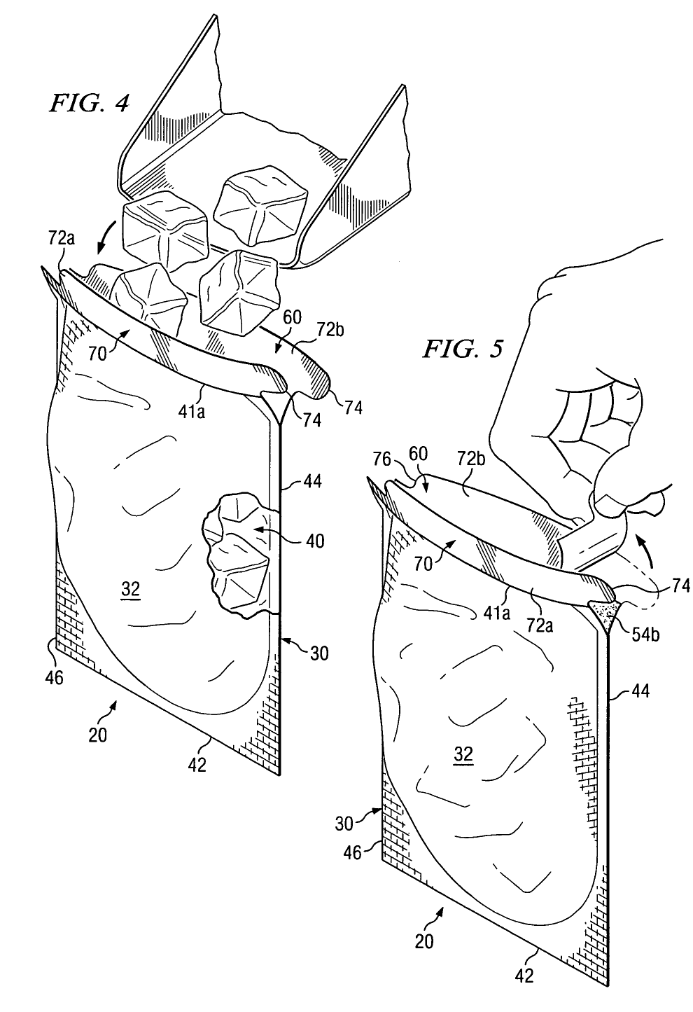

Heat sealing lateral edges 44 and 46 with each other will form a fluid tight barrier with adjacent portions of first layer or inner layers 31. The fluid tight barrier will terminate at the edge of release liners 72a and 72b. As a result, a small or slight gap may be formed equal to the combined thickness of release liners 72a and 72b at the junction of lateral edges 54 and 56 indicated as gap 56. See FIGS. 1 and 5.

For some applications release liners 72 may be formed or cut from a generally continuous strip of material (not expressly shown) with very little or no wasted material. For example, a rotary cutting machine may be used to form each release liner 72 having a generally elongated rectangular configuration having a length corresponding approximately with the length of associated adhesive layers 54. The width of each release liner 72 will often be larger than the width of associated adhesive layer 54 to form portions of disposable funnel type device 70. For some applications the width of release liner 72 may correspond approximately with the width of associated adhesive layer 54.

For some applications tab 74 may be formed at one end of each release liner 72. Corresponding notch or recess 76 may be formed in the opposite end of each release liner 72. The dimensions and configuration of tab 74 and notch 76 may be selected to be compatible with each other. As a result, tab 74 of one ice pack 20 may be nested in respective notch 76 of an adjacent ice pack 20. Tabs 74 cooperate with each other to provide a convenient mechanism for removing associated release liners 72a and 72b after ice has been added to containment section 40. Tabs 74 and notches 76 facilitate automated cutting of respective ice packs 20, with little or no material waste, from multiple layers of material disposed in an elongated strip (not expressly shown). The elongated strip may sometimes be referred to as a “blank” or “blank material”.

For some applications multiple ice packs 20 may be fabricated in a substantially automated process using heat sealing dies and rotary cutting elements (not expressly shown). For example, a first elongated sheet of laminated material compatible with forming sidewalls 34b may be placed on a relatively continuous fabrication line. Adhesive material satisfactory for forming adhesive layers 54b may be placed on one side of a first strip of material compatible with forming release liner 72b. Additional adhesive material satisfactory for forming adhesive layers 54a may be placed on one side of a second strip of material compatible with forming release liner 72a. The first strip of release liner and adhesive material may be placed on the first sheet of laminated material proximate the desired location for edges 41b of resulting sidewalls 34b. The second strip of release liner and adhesive material may then be placed on the first strip of release liner with the associated adhesive material located opposite from the first strip of release liner (no adhesive material contact between the first strips and the second strip of release liners). A second elongated sheet of laminated material compatible with forming sidewalls 34a may then be placed on the second strip of release liner with the associated adhesive material displayed proximate the desired location for edges 41a of resulting sidewalls 34a.

The resulting multiple layers of material form an elongated strip or blank. Rollers or other techniques may be used to move the blank through a heat sealing die to form respective fluid barriers 50 in the blank corresponding with the periphery for each resulting bag 30. A rotary cutting element or other similar device may then be used to cut each ice pack 20 (bag 30 and release liners 72a and 72b) from the blank with little or no wasted material.

The formation of bag 30 using heat sealing techniques will preferably cause adhesive material used to form adhesive layers 54a and 54b to flow into adjacent flared portions 52 of fluid barrier 50 to enhance the sealing of any gaps disposed therein. See FIGS. 1 and 5. Placing adhesive layers 54a and 54b adjacent to mouth 60 prior to heat sealing bag 30 improves the resulting fluid tight barrier after release liners 72a and 72b have been removed and adhesive layers 54a and 54b engaged with each other.

Adhesive layers 54a and 54b are generally disposed contiguous with and fill any gaps in adjacent portions of associated fluid barrier 50. For some applications adhesive layers 54a and 54b cooperate with each other to form a gasket type seal between adjacent portions of first layer 31 to form a permanent fluid tight barrier at mouth 60.

Any variations in the configuration and/or dimensions of release liners 72a and 72b, adhesive layers 54a and 54b and/or adjacent portions of fluid barrier 50 will be compensated for by using heat sealing techniques to form fluid barrier 50. Adhesive layers 54a and 54b are preferably formed from viscous material which will flow into and/or fill any gaps. As a result tolerance and dimensional requirements associated with forming mouth 60 and adhesive layers 54a and 54b may be reduced. The design and configuration of bag 30, fluid barrier 50 and release liners 72a and 72b cooperate with each other to allow the use of high speed automated manufacturing techniques to produce ice packs 20.

Release liner 72a and 72b cooperate with each other to form a disposable funnel type device 70 for use in placing ice within containment section 40. See FIG. 4. For some applications operating instructions and/or other information may be preprinted on release liners 72a and 72b. First release liner 72a and second release liner 72b cooperate with each other to prevent ice or water contact with adhesive layers 54a and 54b during filling of containment section 40 with ice.

After containment section 40 has been filled with ice, release liners 72a and 72b may be removed from associated adhesive layers 54a and 54b. See FIG. 5. Release liners 72a and 72b may be placed in a trash receptacle or other suitable disposal facility to further enhance sanitation and cleanliness associated with the use of ice pack 20. Adhesive layers 54a and 54b may then be securely engaged with each other to form a fluid tight barrier at mouth 60 to prevent water leaks from containment section 40.

Although the present invention and its advantages have been described in detail, it should be understood that various changes, substitutions and alterations may be made herein without departing from the spirit and scope of the invention as defined by the following claims.

What is claimed is:

1. An ice pack comprising:
a first sidewall and a second sidewall;
the first sidewall and the second sidewall formed with at least one layer of waterproof material;
the first sidewall and the second sidewall bonded with each other to form a containment section and an opening in communication with the containment section;
the opening defined in part by respective interior portions of the first sidewall and the second sidewall;
at least one adhesive layer disposed on one interior portion of the containment section adjacent to the opening; and
at least one release liner disposed on the at least one adhesive layer.

2. The ice pack of claim 1 further comprising each sidewall having a generally rectangular configuration.

3. The ice pack of claim 1 further comprising:
a first adhesive layer disposed on the interior portion of the first sidewall adjacent to the opening;
a second adhesive layer disposed on the interior portion of the second sidewall adjacent to the opening; and
the first adhesive layer and the second adhesive layer cooperating with each other to form a fluid barrier when the first adhesive layer engages the second adhesive layer.

4. The ice pack of claim 3 further comprising:
a first release liner disposed on the first layer of the adhesive material; and
a second release liner disposed on the second layer of adhesive material.

5. The ice pack of claim 4 further comprising the first release liner and the second release liner cooperating with each other to form a funnel type device.

6. The ice pack of claim 1 wherein each release liner further comprises:
a generally elongated rectangular configuration defined in part by having a first end and a second end;
the first end of the release liner defined in part by a notch;
the second end of the release liner defined in part by an extended tab; and
the configuration of the tab cooperating with the configuration of the notch whereby a series of release liners may be cut from a strip of material.

7. The ice pack of claim 1 wherein the layer of waterproof material selected from the group consisting of polyethylene film, heat melt adhesive film, and waterproof nonwoven material.

8. The ice pack of claim 1 further comprising a second layer of material disposed on the first layer of material.

9. The ice pack of claim 7 further comprising the second layer of material selected from the group consisting from hydroentangled fiber, hydroentangled wood pulp, absorbent polyester compounds, nonwoven fabric, and woven fabric.

10. The ice pack of claim 1 further comprising at least one adhesive layer selected from the group consisting of viscous adhesive material, acrylic based adhesives, rubber based adhesives and adhesive materials capable of filling any gap formed between the first sidewall and the second sidewall adjacent to the mouth.

11. An ice pack comprising:
a first sidewall and a second sidewall;
the first sidewall and the second sidewall formed with at least a first layer of fluid impervious material and a second layer of soft, absorbent material;
the first sidewall and the second sidewall adhesively bonded with each other to form a containment section and an opening in communication with the containment section;
the opening defined in part by respective interior portions of the first sidewall and the second sidewall;
a first adhesive layer disposed on an interior portion of the first sidewall adjacent to the opening;
a second adhesive layer disposed on an interior portion of the second sidewall adjacent to the opening;
a first release liner disposed on the first layer of adhesive material; and
a second release liner disposed on the second layer of adhesive material.

12. The ice pack of claim 11 further comprising:
a first end and a second end with the opening disposed at the first end;
a first edge and a second edge spaced from each other and extending between the first end and the second end; and
the first lateral edge, the second edge and the second end defined in part by adhesive bonds formed on the periphery of the first sidewall and the second sidewall.

13. The ice pack of claim 11 further comprising fluid tight bond disposed along the periphery of the first sidewall and the second sidewall.

14. The ice pack of claim 11 further comprising an enlarged portion of the fluid tight bond disposed adjacent to the opening to accommodate any voids or gaps which may occur when the first adhesive layer engages the second adhesive layer to form a fluid type barrier.

15. The ice pack of claim 11 wherein each sidewall further comprises the layer fluid impervious material laminated with the layer of soft, absorbent material.

16. An ice pack having a containment section and a mouth communicating with the containment section comprising:
a first sidewall and a second sidewall;
the first sidewall and the second sidewall formed with at least one respective layer of waterproof material;
the first sidewall and the second sidewall bonded with each other to form the containment section and the opening to the containment section;
the opening defined in part by respective interior portions of the first sidewall and the second sidewall; and
a one time closure mechanism disposed adjacent to the mouth.

17. The ice pack of claim 18 wherein the one time closure mechanism further comprises:
at least one adhesive layer disposed on one interior portion of the containment section; and
at least one release liner disposed on the one at least one adhesive layer.

18. A method of manufacturing an ice pack comprising:
forming a first sidewall and a second sidewall;
bonding the first sidewall and the second sidewall with each other to form a containment section and an opening communicating with the containment section;
placing a layer of adhesive material on an interior surface of each sidewall assembly adjacent to opening; and
placing a respective release liner on each adhesive layer.

19. The method of claim 18 further comprising forming each sidewall assembly from at least a first layer of material and a second layer of material.

20. The method of claim 18 further comprising forming each released liner from a generally continuous strip of material.

21. The method of claim 18 further comprising:
forming each release liner with a generally elongated rectangular configuration defined in part by a first end and a second end;
forming a notch in the first end of the release liner; and
forming a tab extending from the second end of the release liner with each tab having a configuration corresponding approximately with the associated notch.

22. A method of using an ice pack having a mouth and a containment section comprising:
separating a pair of release liners attached to respective adhesive layer proximate the mouth to form a generally funnel shaped configuration;
placing ice into containment section through the funnel shaped configuration;
removing the release liner from the respective adhesive layers; and
engaging the first adhesive layer with the second adhesive layer to form a fluid barrier between the containment section and exterior portions of the ice pack.
